(19) United States
(12) Patent Application Publication
(10) Pub. No.: US 2014/0163669 A1
(43) Pub. Date: Jun. 12, 2014

(54) DEVICES AND METHODS FOR THE REPLACEMENT OF THE FUNCTIONING OF HEART VALVES

(71) Applicant: MITRALIX LTD., Rehovot (IL)

(72) Inventors: Yonatan BEN-ZVI, Kiryat Tiv'on (IL); Ira YARON, Har Adar (IL)

(73) Assignee: MITRALIX LTD., Rehovot (IL)

(21) Appl. No.: 14/089,980

(22) Filed: Nov. 26, 2013

Related U.S. Application Data
(60) Provisional application No. 61/734,200, filed on Dec. 6, 2012.

(51) Int. Cl. A61F 2/24

(52) U.S. Cl. CPC A61F 2/2412; A61F 2/243
USPC 623/2.11; 623/2.17; 623/2.18

(57) ABSTRACT

A valve device is provided for implantation at the location of a natural heart valve, for replacing the functioning of the natural heart valve. The valve device includes a holding structure and a plurality of leaflets that open and close as the heart beats and pressure changes. The valve device holding structure may comprise a substantially ring-shaped first section, or ventricular ring, and a substantially ring-shaped second section, or atrial ring, connected together by a connector. The valve device holding structure, which may include all parts besides the leaflets themselves, may be formed from a wire or tube made of a suitable flexible material that allows the valve device to be substantially straightened to a low profile in a constrained delivery condition and that allows the valve device to self-expand to its ring-shaped expanded profile when unconstrained during implantation.

### CROSS REFERENCE TO RELATED APPLICATION

The present application claims priority to U.S. provisional application Ser. No. 61/734,200 filed Dec. 6, 2012, the disclosure of which is incorporated herein by reference in its entirety.

### FIELD OF THE INVENTION

The invention relates to devices and methods for the replacement of the functioning of heart valves, in particular the mitral valve.

### BACKGROUND OF THE INVENTION

Heart valves regulate the movement of blood into and out of the chambers of the heart. The mitral valve, positioned between the left atrium and the left ventricle, can be subject to a condition known as mitral regurgitation, in which the mitral valve, for various reasons, does not close properly and some backflow of blood occurs from the left ventricle back into the left atrium. For example, a mitral valve leaflet can experience prolapse during systole, thereby inhibiting leaflet coaptation and permitting backflow of blood into the left atrium.

Various procedures and devices have been proposed to address the condition of mitral regurgitation. For example, some mitral valve repair procedures involve removing a section of a valve leaflet in order to reduce its propensity for prolapse. Other procedures involve mitral valve replacement. The MITRACLIP (Abbott Vascular) is a device intended to be positioned across the mitral valve to create a double orifice, in an effort to allow the valve to close fully during systole.

Despite these efforts, there is a continuing need for improved treatment for mitral valve regurgitation. The various procedures and devices previously proposed can be improved upon in terms of their overall clinical outcome, ease of use, reduction of procedure time and risk, and/or reduction of cost.

### SUMMARY OF THE INVENTION

The present invention provides devices and methods for the replacement of the functioning of heart valves.

A valve device in accordance with the invention provides a valve structure that can be implanted at the location of a natural heart valve, between the atrium and the ventricle, for replacing the functioning of the natural heart valve. The valve device includes a holding structure for holding the valve device in place at the heart valve location and a plurality of leaflets that provide the valve functioning, opening and closing as the heart beats and pressure changes. During diastole, the leaflets are open, allowing blood to flow freely from the atrium to the ventricle, while during systole, the leaflets are closed, preventing backflow from the ventricle to the atrium.

In some embodiments, the valve device holding structure comprises a substantially ring-shaped first section, or ventricular ring, that is adapted to be positioned on the ventricular side of the heart valve location, and a substantially ring-shaped second section, or atrial ring, that is adapted to be positioned on the atrial side of the heart valve location. The holding structure also may comprise a connector located between the first section and the second section, connecting the first section and second section together. One or more valve leaflets are attached to one or both of the first and second sections. The holding structure may further comprise a leaflet frame, wherein the valve leaflets are attached to the leaflet frame, and the leaflet frame is attached to the first section or the second section.

The valve device holding structure, which may include all parts other than the leaflets themselves, may be formed from a wire or tube made of a suitable flexible material that allows the valve device to be substantially straightened to a low profile in a constrained delivery condition and that allows the valve device to self-expand to its ring-shaped expanded profile when unconstrained. For example, the material may be a shape memory material, such as nitinol or another suitable shape memory alloy, or another suitable flexible material such as a suitable metallic material, plastic material and/or composite material

In an implantation method, a valve device as described herein is implanted at the location of the natural heart valve, between the atrium and the ventricle. In some embodiments, the method includes delivering the valve device to the desired location using a delivery system including a delivery catheter. For delivery to the desired location, the valve device is constrained inside the lumen of the delivery catheter in a substantially straightened, reduced-profile delivery condition. For implantation, the delivery catheter is positioned adjacent the heart valve location by a method known in the art. The approach may be, for example, a transseptal approach, a transfemoral approach, a transatrial approach, or a transapical approach. Once the delivery catheter is in position, a deployment element or other suitable component of the delivery system may be used to push, eject or otherwise deploy the valve device from the catheter.

In one sequence of deployment, the delivery catheter approaches the heart valve location from the atrial side. With the outlet of the delivery catheter suitably positioned, the first section is pushed from the delivery device to the ventricular side of the heart valve location, whereby the first section is released and thereby self-expands from its reduced-profile delivery condition to its expanded substantially ring-shaped condition. Using the delivery system, the first section then may be pulled adjacent the heart valve location. After the first section is deployed, and with the outlet of the delivery catheter suitably positioned or repositioned, the second section is then pushed from the delivery device to atrial side of the heart valve location, whereby the second section is released and thereby self-expands from its reduced-profile delivery condition to its expanded substantially ring-shaped condition. The delivery catheter also may enable a parallel deployment of the first and second sections at the same time. With the valve device fully deployed and in position, and thereby implanted, the delivery catheter may then be withdrawn. In alternate embodiments, the delivery catheter approaches the heart valve location from the ventricular side. The second section or atrial ring may be deployed before the first section or ventricular ring is deployed.

Once the valve device is implanted, the first section and second section keep the valve device in place in its implanted position. The first section, which is situated on the ventricular side of the heart valve location, prevents the device from migrating into the atrium, and can preserve a constant force for the device against the native leaflets from the ventricular side of the valve. The second section, which is situated on the atrial side of the heart valve location, prevents the device from migrating into the ventricle, and can preserve a constant force for the device against the native leaflets from the atrial side of the valve. The leaflets are held in position by the holding structure of the valve device, forming a valve between the atrium and the ventricle and thereby replacing the heart valve functioning. The leaflets also may be positioned on either side of native leaflets, meaning the atrial side or the ventricular side.

The overall heart valve device, as well as each of the first section, the second section and optionally the leaflet frame, may be substantially ring-shaped. The term “substantially ring-shaped” means that the device or section, when viewed from the top, substantially forms a ring. The ring may be open or closed, i.e., it need not extend a full 360 degrees around but may be somewhat less than 360 degrees, as long as it provides suitable stability. It also may extend more than 360 degrees around, for example as a coil or spiral with multiple turns. The substantially ring-shaped structure may be substantially circular, substantially “D”-shaped, substantially triangular or polygonal, substantially elliptical, or any other suitable shape, such as a wavy path extending generally around a circle. Each of the substantially ring-shaped first section and the substantially ring-shaped second section may be substantially flat or may have a suitable shape providing a height dimension, for example an undulating or a substantially conical shape.

The first section, the second section and the connector together may be formed of a continuous length of material, such as a wire, strip, rod, tube or bundles or combinations thereof. The use of a bundle of wires or the like can provide the device or a section thereof with high axial strength as well as high flexibility. For example, the use of several thin wires in a twisted bundle or in a braided bundle provides high axial strength and flexibility that can be determined by the twisting or braiding structure. Each end of the wire, strip, rod, tube or bundles or combinations thereof may be rounded, squared-off, pointed, or may have an anchoring element positioned on it. The connector may constitute a segment of the material or a point on the continuous length of material between the first section and the second section. In alternate embodiments, the first section, the second section and the connector may be formed of separate components joined together, with each such component formed of a wire, strip, rod, tube or bundles or combinations thereof.

The connector may take any suitable form; it may be, for example, substantially straight or curved. The valve device may be designed so that, when the valve device is implanted, the connector passes through the middle of the valve orifice, or on either side of the valve commissures. The connector also may pass through one or more of the leaflets. The connector structure may assist in moving both sections towards each other for better positioning.

The leaflets of the valve device may be attached to the first section or to the second section, either directly or by way of a leaflet frame. For example, the valve leaflets may be attached to a leaflet frame that in turn is attached to one of the first section or the second section. The leaflet frame may be used to control the structure of the leaflets after implantation, while allowing the leaflets to be crimped, compressed or otherwise reduced in profile for fitting into a small diameter delivery catheter. The leaflets may be attached to the leaflet frame, or directly to the first section or to the second section, by suturing, sewing, gluing or by any other suitable manner. The leaflets may be attached to a leaflet frame in a manner that insures that the leaflet shape follows the shape of the frame. The leaflets may be created from various materials including natural materials, such as from a bovine, porcine or other animal tissue source, or synthetic or artificial materials. The leaflet production method may include electro-spinning, knitting, plain sheets or another suitable production method.

When the valve device is in its substantially straightened, reduced-profile delivery condition, the leaflets also may be in a reduce-profile delivery condition. For example, the leaflets may be folded, rolled up, wrinkled, or wrapped around the corresponding first or second section or leaflet frame when placed in the constrained position. When transferred from the delivery catheter to the deployed condition, all leaflets open.

In some embodiments, the component to which the leaflets are attached, which may be the first section, the second section, or the leaflet frame, may have an undulating shape, with a series of crests and valleys. The number of crests or valleys can correspond to the number of leaflets. Each leaflet may be positioned with an apex of the leaflet at a crest of the undulating shape, generally between two valleys of the undulating shape, and/or with a centerline generally aligned with a crest of the undulating shape.

### BRIEF DESCRIPTION OF THE DRAWINGS

FIG. 1 shows a perspective view of a first embodiment of a valve device.

FIG. 2 shows the valve device of FIG. 1 with portions of the device straightened.

FIG. 3 shows a perspective view of a second embodiment of a valve device.

FIG. 4 shows a perspective view of a third embodiment of a valve device.

FIG. 5 shows the valve device of FIG. 4 with portions of the device straightened.

FIG. 6 shows a top view of a fourth embodiment of a valve device.

FIG. 7 shows part of the valve device of FIG. 6 with portions of the device straightened.

### DETAILED DESCRIPTION

Certain embodiments of heart valve replacement devices and methods of using them are described herein with reference to the accompanying drawings. These embodiments are only examples, as numerous variations of the invention disclosed herein are possible within the scope of the appended claims.

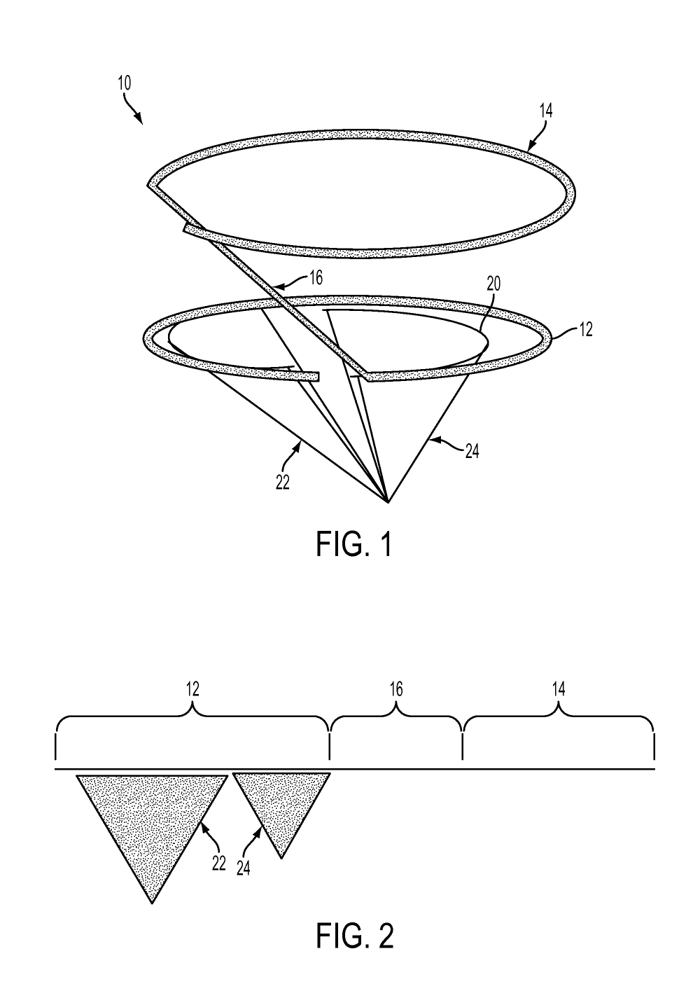

FIG. 1 shows a perspective view of a first embodiment of a valve device, designated as valve device 10. The valve device 10 is substantially ring-shaped. The valve device 10 comprises a substantially ring-shaped first section 12 (or ventricular ring) that is adapted to be positioned on a ventricular side of a heart valve location, a substantially ring-shaped second section 14 (or atrial ring) that is adapted to be positioned on an atrial side of the heart valve location. A connector 16, located between the first section 12 and the second section 14, connects the first section 12 and the second section 14 together. A plurality of valve leaflets 22, 24 are attached to the first section 12 by virtue of the leaflets 22, 24 being attached to a leaflet frame 20 which is attached to the first section 12.

As can be seen in FIG. 1, each of the first section 12 and the second section 14 substantially forms an open ring, i.e., a ring extending less than 360 degrees around the circumference. Alternatively, the first section 12 and/or the second section 14 may extend around the circumference by less or by more than the amount illustrated, such as by more than 360 degrees around, for example as a coil or spiral with multiple turns. The first section 12 and the second section 14 are both illustrated as substantially circular, but they may have any other suitable shape, such as wavy or substantially elliptical. The first section 12 and the second section 14 are both illustrated as substantially flat, but they may have any other suitable shape, for example an undulating or a substantially conical shape.

As can be seen in the view of FIG. 1, the atrial ring 14 can be considered as being wound in a clockwise direction when viewed from the top and starting from the connector 16 and moving outward toward the end of the atrial ring 14. The ventricular ring 12 can be considered as being wound in a counterclockwise direction when viewed from the top and starting from the connector and moving outward toward the end of the ventricular ring 14. Thus, the first section 12 and the second section 14 have windings in opposite directions. In alternative embodiments, the first section and second section may be wound in the same direction.

The connector 16 is illustrated as substantially straight. In alternative embodiments, the connector connecting the first section and the second section may be curved, bent, helical, or any other suitable shape. In one example, the length of the connector may be approximately 1.0-2.0 centimeters (e.g., 1.5 centimeters), but longer or shorter lengths are possible.

The valve device 10 of FIG. 1 is illustrated with two leaflets 22, 24. However, any suitable number of leaflets may be used. For example, three or more leaflets may be used.

The valve device 10 is self-expanding, with its unconstrained profile illustrated in FIG. 1. It can be held in a reduced-profile condition inside of a lumen of a delivery catheter for delivery to the desired area and then released for deployment. Certain portions of the valve device 10 (for example, all parts except the leaflets) may be made of a self-expanding material, such as nitinol or another shape memory material, or any other suitable self-expanding material as is known in the art. In the delivery condition, the entire device 10, including each of the first section 12 and the second section 14, the connector 16, the leaflet frame 20, and the leaflets 22, 24, may be held in a substantially straight shape.

FIG. 2 shows the valve device 10 of FIG. 1 with portions of the device 10 straightened. In particular, the first section 12, the second section 14, and the connector 16 are shown in a straightened condition. The leaflet frame 20 also may be straightened or otherwise changed in profile to fit inside the delivery catheter. The leaflets 22, 24 may be folded, rolled, or wrapped around the first section 12 so that the entire valve device 10 has a substantially straight shape that can fit inside the delivery catheter.

In the valve device 10, the first section 12, the second section 14 and the connector 16 are formed of a continuous length of material. The connector 16 constitutes a segment of that continuous length of material. In alternative embodiments, the connector is simply a point on the continuous length of material between the first section and the second section. In the valve device 10, the first section 12, the second section 14, and the connector 16 are formed of a single wire.

A valve device as described herein may be implanted in a manner similar to the implantation methods described in prior U.S. patent application Ser. No. 13/529,451, filed Jun. 21, 2012. The disclosure of that prior application is hereby expressly incorporated herein by reference.

A method of implanting a valve device, such as valve device 10 or other valve devices described herein, is a follows. The valve device, which is self-expanding, is held in a reduced-profile delivery condition inside a lumen of a delivery catheter of a delivery system. In this reduced-profile delivery condition, the valve device, including the first section and the second section, is held in a substantially straight shape.

With the valve device in the constrained, reduced-profile delivery condition inside the lumen of the delivery catheter, the delivery catheter is positioned adjacent the heart valve location by a method known in the art. The approach may be, for example, a transseptal approach, with the catheter entering the left atrium through the septum between the right atrium and the left atrium. To facilitate a transseptal approach, the delivery system may include an atrial septum dilator. Other approaches alternatively may be used, including, for example, a transfemoral approach through the femoral artery and through the aorta into the left ventricle or a transapical approach through the heart apex into the left ventricle. Once the end of the delivery system is adjacent to the heart valve location, the tip of the delivery system may be moved and/or turned so that it is facing the heart valve location.

In an example approach from the left atrium, the delivery catheter may be advanced through the mitral valve into the left ventricle. The end of the delivery system then may be positioned such that it can deliver the first section of the device on the ventricular side of the heart valve location. Once the end of the delivery catheter is positioned in this manner, the valve device may be delivered from the delivery catheter, such as by a suitable pushing mechanism as is known in the art. Due to the self-expanding or shape memory characteristics of the valve device, once it is delivered from the delivery catheter, the valve device self-expands to its unconstrained shape. Thus, as the first section (or ventricular ring) of the valve device is released from the delivery catheter, the first section self-expands from its reduced-profile delivery condition to assume its expanded, substantially ring-shaped configuration. The catheter or valve device may be turned during delivery of the first section. Once ejected from the delivery catheter, the first section is in position on the ventricular side of the heart valve location. The delivery system then may be used to pull the first section adjacent the heart valve location. After the first section or ventricular ring is delivered, and with the delivery catheter suitably positioned or repositioned, the second section or atrial ring is then pushed from the delivery catheter on an atrial side of the heart valve location. Similar to the delivery of the first section, the delivery of the second section from the delivery catheter releases the second section from its constrained, reduced-profile delivery condition and allows it to self-expand to its substantially ring-shaped condition. The catheter or valve device may be turned during delivery of the second section. The connector may be delivered with the ventricular ring, with the atrial ring, or as a separate step between the deployment of the two rings.

In case a different method of insertion is chosen, e.g. a transapical approach, or for other reasons relating to the desired treatment, the order of the deployment of the sections may change. For example, the second section or atrial ring may be delivered before the first section or ventricular ring is delivered.

In certain embodiments, the ventricular ring is deployed so that it extends around the natural chords. In other embodiments, the ventricular ring is deployed on the inside of the natural chords, so that it pushes the chords outward. In this manner, the ventricular ring can push and hold open the natural valve. In other embodiments, the ventricular ring is deployed on the inside of some chords and on the outside of other chords.

Once the valve device is implanted, the delivery catheter is withdrawn. The valve device remains in place to serve as a valve between the atrium and the ventricle.

Thus, with reference to the embodiment of FIG. 1, when the valve device 10 is suitably implanted, the first section 12 and the second section 14 keep the valve device 10 in place. The first section 12 prevents the device 10 from migrating into the atrium and preserves a constant force for the device 10 against the native leaflets from the ventricular side of the valve. The second section 14 prevents the device 10 from migrating into the ventricle and preserves a constant force for the device 10 against the native leaflets from the atrial side of the valve. The leaflets 22, 24 form a valve between the atrium and the ventricle, replacing the heart valve functioning. In the case of the valve device 10, because the leaflet frame 20 and thus the leaflets 22, 24 are attached to the first section 12, which is the ventricular ring, the implanted valve device 10 has the leaflets 22, 24 positioned on the ventricular side of the heart valve location.

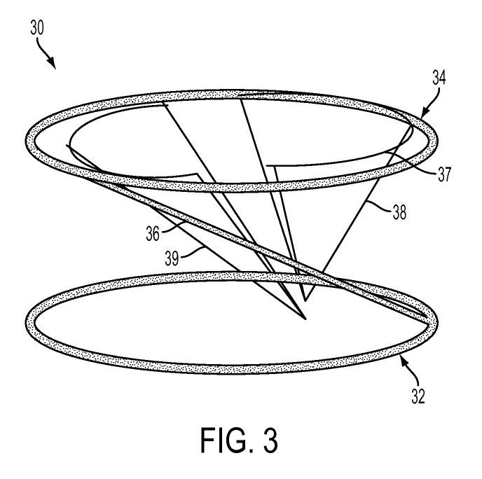

FIG. 3 shows a perspective view of a second embodiment of a valve device, designated as valve device 30. The first section 32, second section 34 and connector 36 are substantially the same as the corresponding parts of valve device 10. However, in the valve device 30, the leaflet frame 37 and the leaflets 38, 39 are attached to the second section 34, which is the atrial ring. The valve device 30 may be implanted in a similar manner as valve device 10. Once implanted, the valve device 30 has the leaflets 38, 39 positioned on the atrial side of the heart valve location.

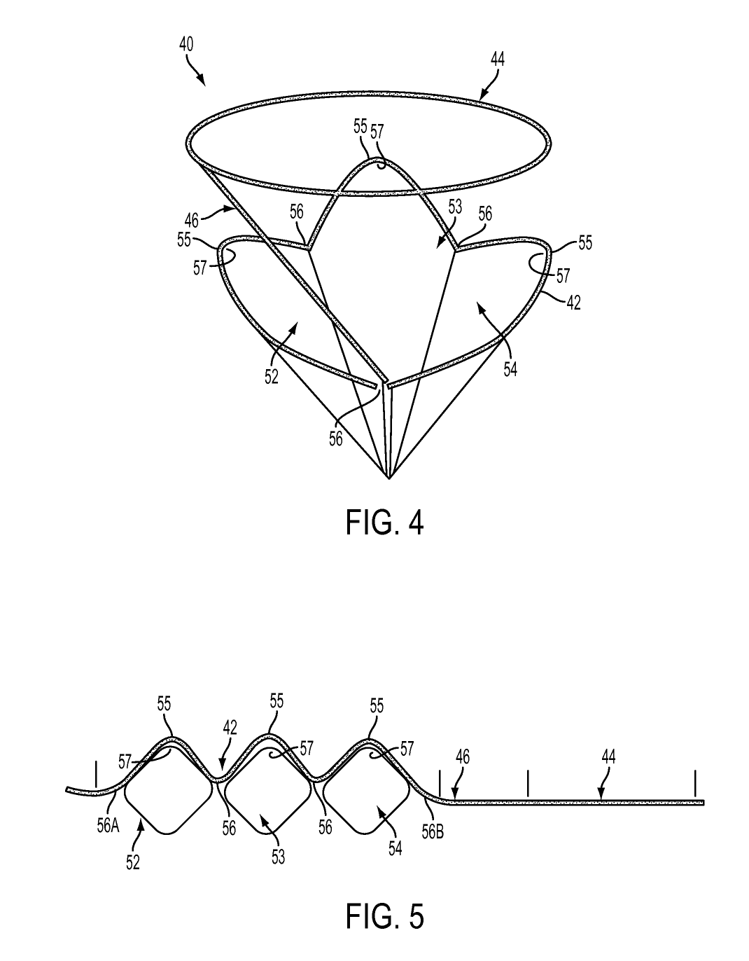

FIG. 4 shows a perspective view of a third embodiment of a valve device, designated as valve device 40. The first section 42, second section 44 and connector 46 are substantially the same as the corresponding parts of valve device 10, except that the first section 42 has an undulating profile for accommodating the valve leaflets 52, 53, 54. Thus, the first section 42 has a series of crests 55 and valleys 56. In the deployed condition, shown in FIG. 4, the crests 55 are closer than the valleys 56 to the second section 44. Each leaflet 52, 53, 54 may be positioned with an apex 57 of the leaflet at a crest 55 of the undulating shape of the first section 42. Each leaflet 52, 53, 54 may be positioned generally between two valleys 56 of the undulating shape of the first section. Each leaflet 52, 53, 54 may have a centerline generally aligned with a crest 55 of the undulating shape of the first section 42. In this illustrated embodiment, the first section 42 has an undulating shape with a series of three crests 55 and three valleys 56, and the valve device 40 correspondingly comprises three leaflets 52, 53, 54, with each leaflet 52, 53, 54 positioned with an apex 57 of the leaflet at a crest 55 of the undulating shape of the first section 42. The undulating profile provides additional support for the leaflets 52, 53, 54. The leaflets 52, 53, 54 are adjacent to the first section 42 over a substantial portion of their perimeter and may be attached to the first section 42 along much or all of this adjacent length.

FIG. 5 shows the valve device 40 of FIG. 4 with portions of the device straightened. In particular, the first section 42, the second section 44, and the connector 46 are shown in a straightened condition. The leaflets 52, 53, 54 may be folded, rolled, or wrapped around the first section 42 so that the entire valve device 40 has a substantially straight shape that can fit inside the delivery catheter. It will be appreciated from a comparison of FIG. 5 to FIG. 4 that points 56A and 56B in FIG. 5 come together in the deployed configuration of FIG. 4 to form one of the valleys 56.

The valve device 40 may be implanted in a similar manner as valve device 10. Once implanted, the valve device 40 has the leaflets 52, 53, 54 positioned on the ventricular side of the heart valve location. In an alternative embodiment, the second section may have an undulating shape and the leaflets may be attached to the second section. Once implanted, such a valve device has the leaflets positioned on the atrial side of the heart valve location.

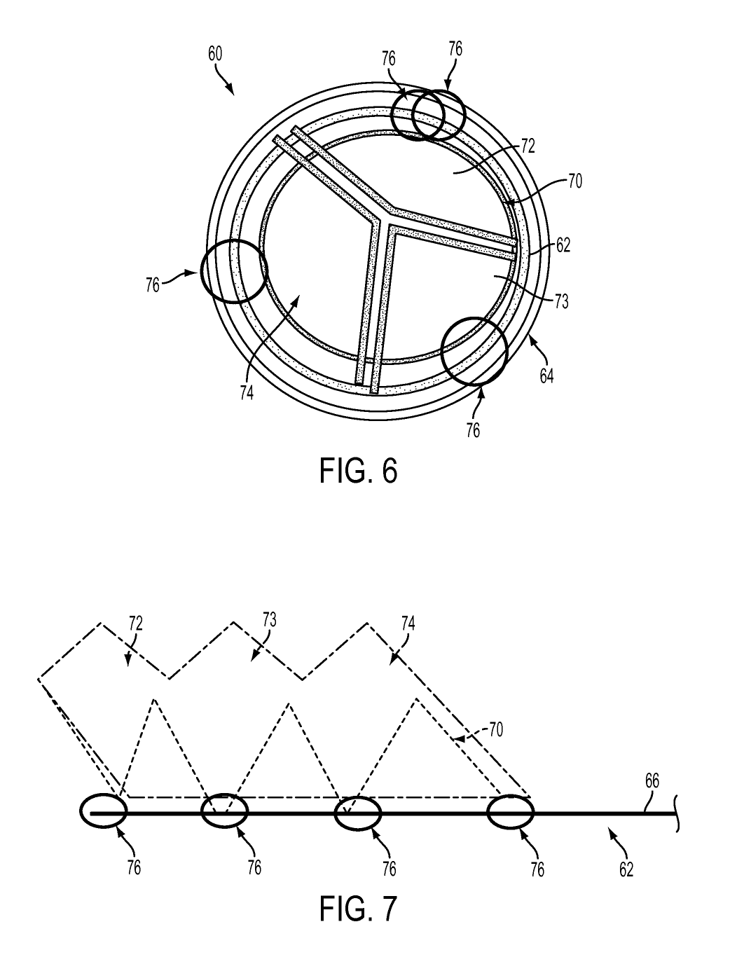

FIG. 6 shows a top view of a fourth embodiment of a valve device, designated as valve device 60. FIG. 7 shows part of the valve device 60 of FIG. 6 with portions of the device straightened.

The valve device 60 is similar in many respects to the valve devices already described. The valve device 60 comprises a first section (ventricular ring) 62, a second section (atrial ring) 64, and a connector 66 connecting the first section 62 and the second section 64. The valve device 60 further comprises a leaflet frame 70 and a plurality of leaflets 72, 73, 74. The valve device 60 further comprises four connection rings 76 for connecting the leaflet frame 70 to the first section 62.

The valve device 60 is similar to the valve device 10. However, the leaflet frame 70 in valve device 60 has an undulating profile for accommodating the valve leaflets 72, 73, 74, and the leaflet frame 70 is connected to the first section 62 by the connection rings 76. Thus, the leaflet frame 70 has a series of crests and valleys, similar to the undulating first section 42 of valve device 40. In the deployed condition, the crests of the leaflet frame 70 are closer than the valleys of the leaflet frame 70 to the second section 64. Each leaflet 72, 73, 74 may be positioned with an apex of the leaflet at a crest of the undulating shape of the leaflet frame 70. Each leaflet 72, 73, 74 may be positioned generally between two valleys 76 of the undulating shape of the leaflet frame 70. Each leaflet 72, 73, 74 may have a centerline generally aligned with a crest of the undulating shape of the leaflet frame 70. The undulating profile of the leaflet frame provides additional support for the leaflets 72, 73, 74. The leaflets 72, 73, 74 are adjacent to the leaflet frame 70 over a substantial portion of their perimeter and may be attached to the leaflet frame 70 along much or all of this adjacent length.

Various modifications of the illustrated and described embodiments may be made within the scope of the claims.

For example, the cross-section of the wire, strip, rod, tube or bundles or combinations thereof that form all or part of the holding structure may be circular, elliptical, rectangular or any other suitable shape. In one example, the wire, strip, rod, tube or bundle or combination may have a diameter, width or thickness of approximately 0.2-1.0 millimeters (e.g., 0.4 millimeters), but larger or smaller dimensions are possible. All or part of the holding structure may be formed by bending or otherwise shaping the wire, strip, rod, tube or bundle or combination thereof into the desired shape. Alternatively, the shape may be formed as the wire, strip, rod, tube or bundle or combination thereof is formed. For example, the shape of the first section and/or the second section may be chemically or laser etched or otherwise cut from a sheet of material, in which case the strip or rod is formed simultaneously with the section itself. The holding structure may be formed of more than a single structure or material; for example, a tube with a wire core may form the upper section (atrial ring), the lower section (ventricular ring) and/or the connector between them, with the other element(s) formed of a similar or dissimilar structural component.

In some embodiments, the wire, strip, rod, tube or bundle or combination may have one or more holes in it. The holes may be through-holes extending all the way through the thickness of the wire, strip, rod, tube, bundle or combination, and/or the holes may be pockets or dimples in the outer surface of the wire, strip, rod or tube, bundle or combination. The holes may be a series of holes extending along the length and/or around the periphery of the wire, strip, rod or tube, bundle or combination. The holes may serve different purposes. For example, one or more holes may be used to create different flexibilities at different places of the device, to facilitate ingrowth of tissue, to facilitate grasping and manipulation of the device, to provide ports for injection of a contrast agent, and/or to serve as sites for drug delivery.

If the valve device or a portion thereof is formed as a tube, a wire or stiffening element may be placed into the tube in order to change the stiffness and/or shape of the tube or a section of it. For example, a stiffening element may be used to maintain the valve device in a first shape for delivery (e.g., relatively straight), and the stiffening element may be withdrawn upon delivery of the valve device from the delivery catheter in order to allow the valve device to take its implantation shape. In another example, an inner wire may be attached to the distal end of the tube, and the inner wire may be pulled relative to the tube to change the shape of the tube. Pulling the inner wire applies a compressive force to the tube. The tube may be formed with pre-shaped side cuts along the tube, such that it bends in a predetermined pattern, e.g., into a substantially ring-shaped pattern, when such a load is applied. A locking mechanism may be used to lock the wire in its loaded position relative to the tube. Different depths and widths of the side cuts and the distance between the side cuts would determine the final shape of the tube element once a load is applied.

The valve device may comprise a coating, for example, on the wire, strip, rod, tube or bundles or combinations thereof that form all or part of the holding structure, or on the leaflets. The coating is preferably a biocompatible coating that may be used, for example, to reduce possible negative reactions from the tissue where the valve device is implanted, to reduce friction (as a lubricious coating) to assist in delivery of the valve device, to reduce friction in areas where the valve device is designed to be moved against tissue, to increase friction in areas where it is desired to reduce movement or to anchor the device, to deliver a suitable drug, for radiopacity, to encourage cell and tissue growth that would assist in fixation (e.g., of the upper section or atrial ring), to encourage tissue growth between the chords and/or leaflets, and/or for other purposes. With respect to radiopacity, the entire device or selected points on the device may be coated or plated with a material allowing the physician to understand the location of the device during and/or after the implantation procedure.

The valve device may have one or more anchoring elements for anchoring the valve device to heart tissue. For example, one or more barbs, hooks, loops, sutures, etc., may be provided on the holding structure, or sutures or staples may be applied at selected locations. The anchoring may have various functions: anchoring the valve device to the native valve in various places, anchoring the holding structure to the new leaflets, and connecting the ends of the atrial ring or the ventricular ring after deploying from the straightened condition to the substantially ring-shaped deployed condition.

In certain embodiments, more than one valve device may be used, with each valve device having one or more leaflets as described before. Therefore, the implantation method, or parts of it, may be repeated several times in order to place all of the new leaflets in place.

The valve device may have other elements to monitor the functioning of the device. For example, the device may be equipped with a sensor attached to the device. The sensor may be, for example, a pressure sensor, a temperature sensor, and/or a velocity sensor. In this way, the operation of the valve and the blood flow can be monitored. Similarly, the device itself when formed as a tube can be used as a “pig tail” for measuring pressure during or after the implantation procedure.

In one example of the use of sensors, the use of MEMS (microelectromechanical systems) sensors on the device may assist in the implantation procedure or during the years after it. Such sensors may monitor temperature, oxygen saturation, pressure, blood velocity or similar physical characteristics. During the implantation procedure, it is possible to use an xyz (positioning) sensor on the device to assist in the accurate location and positioning of the device by using an external system that reads the information transmitted from the sensor.

Sensor(s) on the device or delivery system may be part of a closed-loop system that uses the signals from the sensor(s) as feedback for automatic delivery and positioning. By using pressure sensors in the ventricle and atrium, the pressure can be continuously monitored as the device is automatically adjusted. The adjustments and monitoring can be continued until target pressure readings are achieved. This automatic positioning with the use of feedback can eliminate the need for manual monitoring and positioning that can be complicated and less accurate.

The device also may have an energy-producing element that produces energy by the flow of blood around the device and/or by the pressure changes using a converter (such as a piezoelectric element that is capable of converting mechanical pulse into electric current). The energy may charge a battery that, for example, can be used to transmit signals from one or more sensors as described above.

Based on the above description and the accompanying drawings, the principles and operation of the invention, as well as how to make and use the invention, can be understood by persons of ordinary skill in the art. Many embodiments and variations are possible that take advantage of the principles and operation of the invention described herein. The examples described herein and shown in the accompanying drawings are meant as examples only and are not intended to be limiting of the scope of the invention defined by the appended claims.

What is claimed is:

1. A valve device for replacing the functioning of a heart valve comprising:
a first section that is substantially ring-shaped and adapted to be positioned on a ventricular side of a heart valve location;
a second section that is substantially ring-shaped and adapted to be positioned on an atrial side of the heart valve location;
a connector located between the first section and the second section and connecting the first section and the second section together; and
one or more valve leaflets attached to one of the first section or the second section.

2. A valve device as in claim 1, comprising two or more valve leaflets.

3. A valve device as in claim 2, wherein the valve device has a self-expanding construction.

4. A valve device as in claim 3, wherein the valve device is deformable to a constrained, reduced-profile delivery condition in which the valve device fits inside a lumen of a delivery catheter, and wherein the valve device is self-expandable to an expanded deployment condition upon deployment from the delivery catheter.

5. A valve device as in claim 4, wherein in the reduced-profile delivery condition the first section is held in a substantially straight shape and the second section is held in a substantially straight shape.

6. A valve device as in claim 2, wherein the valve leaflets are attached to the first section.

7. A valve device as in claim 2, wherein the valve leaflets are attached to the second section.

8. A valve device as in claim 2, wherein the section to which the valve leaflets are attached has an undulating shape, and wherein each valve leaflet is positioned with an apex of the undulating shape.

9. A valve device as in claim 2, wherein the valve device further comprises a leaflet frame, wherein the valve leaflets are attached to the leaflet frame, and the leaflet frame is attached to one of the first section or the second section.

10. A valve device as in claim 9, wherein the leaflet frame has an undulating shape, and wherein each valve leaflet is positioned with an apex of the undulating shape.

11. A valve device as in claim 1, wherein the first section is substantially flat.

12. A valve device as in claim 1, wherein the second section is substantially flat.

13. A valve device as in claim 1, wherein the first section and the second section are formed of a continuous length of material.

14. A valve device as in claim 1, wherein the first section, the second section and the connector are formed of a continuous length of material.

15. A valve device as in claim 1, wherein the valve device is comprised at least in part of a shape memory metal material.

16. A method of implanting a valve device for replacing the functioning of a heart valve comprising:
delivering a valve device to a heart valve location, the valve device comprising a first section that is substantially ring-shaped and adapted to be positioned on a ventricular side of the heart valve location, a second section that is substantially ring-shaped and adapted to be positioned on an atrial side of the heart valve location, a connector located between the first section and the second section and connecting the first section and the second section together, and a plurality of valve leaflets attached to one of the first section or the second section;
positioning the first section on a ventricular side of the heart valve location; and
positioning the second section on an atrial side of the heart valve location;
whereby the leaflets form a valve between the atrium and the ventricle.

17. A method as in claim 16, wherein during the step of delivering the valve device to the area of the heart valve location, the valve device is held in a reduced-profile delivery condition inside a lumen of a delivery catheter.

18. A method as in claim 17, wherein the step of positioning the first section on a ventricular side of the heart valve location comprises pushing the first section from the delivery device, whereby the first section is released from its reduced-profile delivery condition and self-expands to its substantially ring-shaped condition.

19. A method as in claim 17, wherein the step of positioning the second section on an atrial side of the heart valve location comprises pushing the second section from the delivery device, whereby the second section is released from its reduced-profile delivery condition and self-expands to its substantially ring-shaped condition.

20. A method as in claim 17, wherein in the delivery condition the first section is held in a substantially straight shape and the second section is held in a substantially straight shape.
